(19) United States
(12) Patent Application Publication
(10) Pub. No.: US 2005/0060027 A1
(43) Pub. Date: Mar. 17, 2005

(54) Catheter balloon systems and methods

(75) Inventors: Michael Khenansho, Modesto, CA (US); Eric Williams, Fairfield, CA (US); Daryush Mirzaee, Sunnyvale, CA (US); Michael Schwartz, San Francisco, CA (US); Charles J. Davidson, Winnetka, IL (US); Gil M. Vardi, Town and Country, MO (US); Amnon Yadin, Pleasanton, CA (US)

Correspondence Address: PATTON BOGGS LLP, 8484 WESTPARK DRIVE, SUITE 900, MCLEAN, VA, 22102, US

(73) Assignee: Advanced Stent Technologies, Inc., Pleasanton, CA (US)

(21) Appl. No.: 10/893,278

(22) Filed: Jul. 19, 2004

Related U.S. Application Data
(63) Continuation-in-part of application No. 10/834,066, filed on Apr. 29, 2004.
(63) Continuation-in-part of application No. 10/802,036, filed on Mar. 17, 2004.
Application No. 10/802,036 is a continuation-in-part of application No. 10/705,247, filed on Nov. 12, 2003.
Application No. 10/802,036 is a continuation-in-part of application No. 09/668,687, filed on Sep. 22, 2000, now abandoned.
Application No. 09/668,687 is a continuation-in-part of application No. 09/326,445, filed on Jun. 4, 1999, now Pat. No. 6,325,826.
Application No. 10/705,247 is a continuation-in-part of application No. 10/440,401, filed on May 19, 2003.
Application No. 10/440,401 is a continuation of application No. 09/750,372, filed on Dec. 27, 2000, now Pat. No. 6,599,316.
Application No. 10/440,401 is a continuation-in-part of application No. 09/963,114, filed on Sep. 24, 2001, now Pat. No. 6,706,062.
Application No. 09/963,114 is a continuation of application No. 09/326,445, filed on Jun. 4, 1999, now Pat. No. 6,325,826.
Application No. 09/326,445 is a continuation-in-part of application No. PCT/US99/00835, filed on Jan. 13, 1999.
(63) Continuation-in-part of application No. 10/644,550, filed on Aug. 21, 2003.
(60) Provisional application No. 60/488,006, filed on Jul. 18, 2003.
(60) Provisional application No. 60/518,870, filed on Nov. 12, 2003.
(60) Provisional application No. 60/547,778, filed on Feb. 27, 2004.
(60) Provisional application No. 60/548,868, filed on Mar. 2, 2004.
(60) Provisional application No. 60/404,756, filed on Aug. 21, 2002.
(60) Provisional application No. 60/487,226, filed on Jul. 16, 2003.
(60) Provisional application No. 60/488,006, filed on Jul. 18, 2003.
(60) Provisional application No. 60/488,006, filed on Jul. 18, 2003.

(51) Int. Cl. A61F 2/06

(52) U.S. Cl. 623/1.35

(57) ABSTRACT

A system for treatment of a bifurcation of a body lumen, the bifurcation having a main vessel and a branch vessel, the system includes a catheter having a main catheter shaft and a first balloon associated with the main catheter shaft, a side sheath and a second balloon associated with the side sheath, and a stent including a generally cylindrical body and a branch portion. A method is also described which includes advancing a catheter system through the main vessel, positioning a branch portion of a stent present in the system proximate to a branch vessel, and inflating first and second balloons thereby expanding a main body and branch portion of the stent.

The present application in a Continuation-in-Part of co-pending U.S. patent application Ser. No. 10/834,066, filed Apr. 29, 2004, which claims the benefit of priority of U.S. Provisional Application No. 60/488,006 filed Jul. 18, 2003; U.S. Provisional Application No. 60/518,870 filed Nov. 12, 2003; U.S. Provisional Application No. 60/547,778 filed Feb. 27, 2004; and U.S. Provisional Application No. 60/548,868 filed Mar. 2, 2004. The present application is also a Continuation-in-Part of co-pending U.S. patent application Ser. No. 10/802,036, filed Mar. 17, 2004, which is, in turn, a Continuation-in-Part of co-pending U.S. patent application Ser. No. 10/705,247, filed Nov. 12, 2003, and is a Continuation-in-Part of co-pending U.S. application Ser. No. 09/668,687, filed Sep. 22, 2000, which is a Continuation-in-Part of U.S. patent application Ser. No. 09/326,445, filed Jun. 4, 1999, now U.S. Pat. No. 6,325,826, and is a Continuation-in-Part of co-pending U.S. patent application Ser. No. 10/440,401, filed May 19, 2003, which is a Continuation of U.S. patent application Ser. No. 09/750,372, filed Dec. 27, 2000, now U.S. Pat. No. 6,599,316, and is a Continuation-in-Part of U.S. patent application Ser. No. 09/963,114, filed Sep. 24, 2001, now U.S. Pat. No. 6,706,062, which is a Continuation of U.S. patent application Ser. No. 09/326,445, filed Jun. 4, 1999, now U.S. Pat. No. 6,325,826, which is a Continuation-in-Part of International Application No. PCT/US99/00835, filed Jan. 13, 1999. The present application is also a Continuation-in-Part of co-pending U.S. patent application Ser. No. 10/644,550 filed Aug. 21, 2003, which claims the benefit of priority to U.S. Provisional Application No. 60/404,756 filed Aug. 21, 2002, U.S. Provisional Application No. 60/487,226 filed Jul. 16, 2003, and U.S. Provisional Application No. 60/488,006 filed Jul. 18, 2003. The present application claims the benefit of priority of U.S. Provisional Application No. 60/488,006, filed Jul. 18, 2003. The complete disclosures of the above-referenced applications are incorporated herein by reference.

### FIELD OF THE INVENTION

The present invention relates to the field of medical balloon catheters and, more particularly, to systems for delivering a stent at or near a bifurcation of a body lumen.

### BACKGROUND OF THE INVENTION

Balloon catheters, with or without stents, are used to treat strictures, stenoses, or narrowings in various parts of the human body. Devices of numerous designs have been utilized for angioplasty, stents and grafts or combination stent/grafts. Varied catheter designs have been developed for the dilatation of stenoses and to deliver prostheses to treatment sites within the body lumen.

Illustrative procedures involving balloon catheters include percutaneous transluminal angioplasty (PTA) and percutaneous transluminal coronary angioplasty (PTCA), which may be used to reduce arterial build-up such as caused by the accumulation of atherosclerotic plaque. These procedures involve passing a balloon catheter over a guidewire to a stenosis with the aid of a guide catheter. The guidewire extends from a remote incision to the site of the stenosis, and typically across the lesion. The balloon catheter is passed over the guidewire, and ultimately positioned across the lesion.

Once the balloon catheter is positioned appropriately across the lesion, (e.g., under fluoroscopic guidance), the balloon is inflated, which breaks the plaque of the stenosis and causes the arterial cross section to increase. Then the balloon is deflated and withdrawn over the guidewire into the guide catheter, and from the body of the patient.

In many cases, a stent or other prosthesis must be implanted to provide support for the artery. When such a device is to be implanted, a balloon catheter which carries a stent on its balloon is deployed at the site of the stenosis. The balloon and accompanying prosthesis are positioned at the location of the stenosis, and the balloon is inflated to circumferentially expand and thereby implant the prosthesis. Thereafter, the balloon is deflated and the catheter and the guidewire are withdrawn from the patient.

Administering PTCA and/or implanting a stent at a bifurcation in a body lumen poses further challenges for the effective treatment of stenoses in the lumen. For example, dilating a main vessel at a bifurcation may cause narrowing of the adjacent branch vessel. In response to such a challenge, attempts to simultaneously dilate both branches of the bifurcated vessel have been pursued. These attempts include deploying more than one balloon, more than one prosthesis, a bifurcated prosthesis, or some combination of the foregoing. However, simultaneously deploying multiple and/or bifurcated balloons with or without endoluminal prostheses, hereinafter individually and collectively referred to as a bifurcated assembly, requires accurate placement of the assembly. Deploying multiple stents requires positioning a main body within the main vessel adjacent the bifurcation, and then attempting to position another stent separately into the branch vessel of the body lumen. Alternatives to that include deploying a dedicated bifurcated stent including a tubular body or trunk and two tubular legs extending from the trunk. Examples of bifurcated stents are shown in U.S. Pat. No. 5,723,004 to Dereume et al., U.S. Pat. No. 4,994,071 to MacGregor, and U.S. Pat. No. 5,755,734 to Richter et al.

Additional bifurcation stent delivery systems that provide improved reliable treatment at bifurcations are disclosed, for example, in U.S. Pat. No. 6,325,826 to Vardi et al. and U.S. Pat. No. 6,210,429 to Vardi et al. The contents of these aforementioned patents are incorporated herein by reference.

A need still exists for further improved devices and techniques for treating a bifurcated body lumen. For example, a need further exists for additional stent delivery systems that can be used with stents having a branch access side hole and/or an extendible branch portion, of the type disclosed in U.S. Pat. No. 6,210,429.

### SUMMARY OF THE INVENTION

The present invention is directed to devices and techniques for treating a bifurcated body lumen including systems for delivering an endoluminal prosthesis at or near a bifurcation of a body lumen. Systems, devices and techniques are disclosed comprising balloon catheters configured to successfully and reliably deploy stents at a bifurcation in a body lumen. Additionally, the balloon catheters can be employed as balloon angioplasty catheters to treat occlusions in blood vessels such as for instance in percutaneous transluminal coronary angioplasty (PTCA) procedures.

According to one aspect, the present invention provides a system for treatment of a bifurcated body lumen, the bifurcated body lumen comprising a main vessel and a branch vessel, the system comprising: a catheter comprising a main catheter shaft and a first balloon associated with the main catheter shaft; a side sheath and a second balloon associated with the side sheath; and a stent comprising a generally cylindrical body defining an outer perimeter having a proximal end and a distal end and a branch portion; wherein the stent is positioned relative to the side sheath such that the first balloon is adapted to expand the main body portion of the stent, and the second balloon is adapted to extend the branch portion toward the branch vessel, and wherein the second balloon is located radially inward of the outer perimeter when the second balloon is not inflated.

According to another aspect, the present invention provides a system for treatment of a bifurcated body lumen, the bifurcated body lumen comprising a main vessel and a branch vessel, the system comprising: a catheter comprising a main catheter shaft and a first balloon associated with the main catheter shaft; a side sheath and a second balloon associated with the side sheath; and a stent comprising a generally cylindrical body having a proximal end and a distal end, a branch portion, and a branch access opening; wherein the start is positioned relative to the side sheath such that the first balloon is adapted to expand the main body portion of the stent, and the second balloon is adapted to extend the branch portion toward the branch vessel, and the second balloon is longitudinally located between the proximal end and the distal end of the stent; and wherein at least a portion of the side sheath extends through the branch access opening.

According to yet another aspect, the present invention provides a method for treating a bifurcated body lumen, the bifurcated body lumen comprising a main vessel and a branch vessel, the method comprising: (i) advancing a catheter system through the main vessel, the catheter system comprising: a main catheter shaft and a first balloon associated with the main catheter shaft; a side sheath and a second balloon associated with the side sheath; and a stent comprising a generally cylindrical body having a proximal end, a distal end, a branch portion, and a branch access opening; wherein at least a portion of the side sheath extends through the branch access opening; and wherein the second balloon is longitudinally located between the proximal end and the distal end of the stent; (ii) positioning the branch portion of the stent proximate to the branch vessel; (iii) inflating the first balloon thereby causing expansion of the generally cylindrical body of the stent; and (iv) inflating the second balloon thereby causing the branch portion of the stent to be pushed outward with respect to the generally cylindrical body of the stent.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented to provide what is believed to be the most useful and readily understood description of the principles and conceptual aspects of the invention.

FIG. 1 is a side view of an illustrative embodiment of a stent delivery system constructed in accordance with the present invention.

FIG. 2 is an enlarged side view taken of the distal portion of the system of FIG. 1.

FIG. 3 is a view of the stent delivery system of FIG. 1 in a blood vessel shown approaching a bifurcation in the vessel without a stent mounted thereon in accordance with a method of the present invention.

FIG. 4 is a view of the system of FIG. 3, including a stent mounted thereon.

FIG. 5 is a view of the stent delivery system of FIG. 1 in a blood vessel located at a bifurcation in the vessel without a stent mounted thereon in accordance with a method of the present invention.

FIG. 6 is a cross-sectional side view of the stent delivery system of FIG. 1 with a stent mounted thereon and shown in the expanded condition.

FIG. 7 is a perspective view of a balloon configured according to one embodiment of the present invention.

FIG. 8 is a perspective view of a balloon constructed according to an alternative embodiment of the present invention.

FIG. 9 is a perspective view of a balloon configured according to a further embodiment of the present invention.

FIG. 10 is a perspective view of a balloon configured according to yet another alternative embodiment of the present invention.

FIG. 11 is a perspective view of a balloon configured according to another embodiment of the present invention.

FIG. 12 is a flat view of another embodiment of an unexpanded stent in accordance with the present invention.

FIG. 13 is a perspective view of the expandable branch portion of the stent of FIG. 12 in the expanded configuration.

FIG. 14 is a flat view of another embodiment of an unexpanded stent in accordance with the present invention.

FIG. 15 is an enlarged view of a portion of the stent of FIG. 14.

FIG. 16 is a view of the expandable branch portion of the stent of FIG. 14 in the expanded configuration.

FIG. 17 is a flat view of another embodiment of an unexpanded stent in accordance with the present invention.

FIG. 18 is a flat view of another embodiment of an unexpanded stent in accordance with the present invention.

FIG. 19 is a view of an expandable branch portion of the stent of FIG. 18 in the expanded condition.

FIG. 20 is a schematic view of a stent in the expanded state implemented at a blood vessel bifurcation.

FIG. 21 is a schematic view of the stent of an alternative construction in the expanded state implemented at a blood vessel bifurcation.

FIG. 22 is a perspective view of an alternative stent delivery system for inserting a stent in accordance with another system and method of the present invention.

FIGS. 23-26 are illustrations of the steps for a method of inserting a stent according to one embodiment of the present invention.

### DESCRIPTION OF THE PREFERRED EMBODIMENTS

The present invention relates to balloon catheters such as balloon angioplasty catheters to treat occlusions in blood vessels. The balloon catheters can be used alone or with a stent, prosthesis or graft. Such a stent delivery system can be used for placement of a stent in a body lumen, particularly at vessel bifurcations. A preferred stent to be delivered is generally configured to at least partially cover a portion of a branch vessel as well as a main vessel. In general, a wide variety of stents and deployment methods may be used with the stent delivery system embodiments of the present invention and the present invention should be understood to not be limited to any particular stent design or configuration. Examples of the types of stents that may be used with the delivery systems of the present invention are disclosed, for example, in U.S. Pat. No. 6,210,429 to Vardi et al., U.S. Pat. No. 6,325,826 to Vardi et al., co-pending U.S. patent application Ser. No. 10/802,036 entitled “Stent With Protruding Branch Portion For Bifurcated Vessels,” and co-pending U.S. patent application Ser. No. 10/644,550, entitled “Stent With a Protruding Branch Portion For Bifurcated Vessels,” the entire contents of which are incorporated herein by reference. In general, the aforementioned stents include a branch portion located at some point along the length of the stent that is configured to be extendible into a branch vessel in a vessel bifurcation. Once the stent is in position in the main vessel and the branch portion is aligned with the side branch vessel the stent can be expanded and the delivery system is particularly adapted to expand the stent branch portion into the side branch vessel. The stent, including the branch portion, may be expanded with a single expansion or multiple expansions as disclosed, for example, in co-pending U.S. patent application Ser. No. 10/834,066, the entire content of which is incorporated by reference.

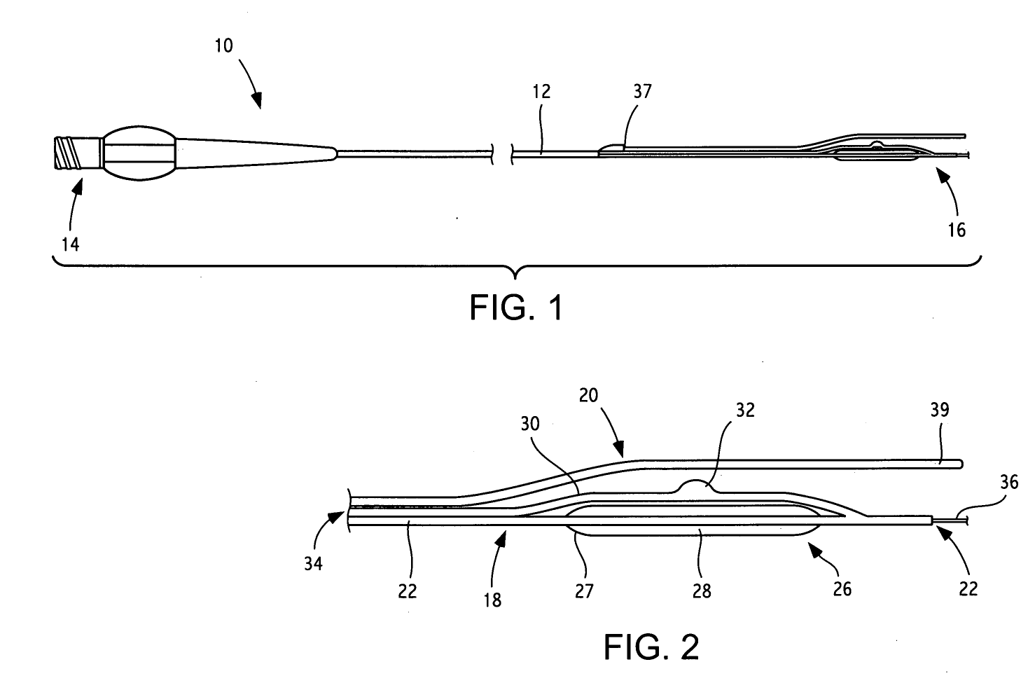

An illustrative view of one embodiment of a stent delivery system 10 constructed in accordance with the present invention is shown in FIG. 1. Stent delivery system 10 generally comprises an elongate main catheter shaft 12 extending from a proximal end 14 to a distal end 16. As best seen in FIG. 2, distal end 16 has a bifurcated tip structure with two branch portions, a main vessel branch portion 18 and a side branch sheath 20 that branch off of main catheter shaft 12. A bifurcated balloon 26 is attached to main vessel branch portion 18 adjacent the distal end 16 and comprises first and second branch portions 27, 30. First branch portion 27 of balloon 26 comprises an elongate inflatable portion 28. Second branch portion 30 of balloon 26 comprises a second or auxiliary balloon or inflatable portion 32. Second branch portion 30 includes an inflation lumen that branches off from first branch portion 27 proximally from the balloon 26 and extends substantially adjacent elongate inflatable portion 28. The distal end of second branch portion 30 is attached to first branch portion 27 at a location distally from the balloon 26. In one preferred embodiment, the distal end of branch portion 30 is fixedly attached distally of balloon 26 in order to prevent at least the second inflatable portion 32 from moving around the first branch portion 27, although in alternate embodiments it may be removably attached.

In a first embodiment, first inflatable portion 28 is generally cylindrical and extends coaxially along main vessel branch portion 18. Second inflatable portion 32 may have a shape and size adapted to extend into the branch vessel as shown and described herein. For example, portion 32 may have a generally offset configuration and may be positioned adjacent or in abutting relation with respect to elongate inflatable portion 28.

The first and second inflatable portions or balloons can have varied shapes, sizes and positioning in accordance with the principles of the invention. For example, in alternative design variations, accurate sizing and positioning of the inflatable portions relative to the vessel may be achieved.

According to the present invention, the inflatable portions, or balloons, can be constructed of any suitable material. For example, the balloons may be constructed of an appropriate polymeric material. Particular examples include the polyamide family, or the polyamide blend family, polyethylene (PE), polyethylene terephthalate (PET), polyurethanes, polyamides, and polyamide blends such as PBAX. The compliance of the first inflatable portion 28 and the second inflatable portion 32 can be the same or different. In one preferred embodiment, second inflatable portion 32 is longitudinally positioned at a generally central location relative to the first inflatable portion 28. In alternate embodiments, second inflatable portion 32 may be positioned at any position adjacent first inflatable portion 28.

In a preferred embodiment, balloon branch portions 27 and 30 have a common inflation lumen 34. Inflation lumen 34 can be conventional, and extend from a portion of the stent delivery system which always remains outside of the patient (not pictured). Inflation lumen 34 extends distally into each of first and second branch portions 27 and 30 and thus, inflation lumen 34 is in fluid communication with the interiors of first inflatable portion 28 and second inflatable portion 32. Thus inflation lumen 34 is used to supply pressurized inflation fluid to first inflatable portion 28 and second inflatable portion 32 when it is desired to inflate balloon 26. Inflation lumen 34 is also used to drain inflation fluid from first inflatable portion 28 and second inflatable portion 32 when it is desired to deflate the balloon. First and second inflatable portions are initially deflated when directing the stent delivery device to the bifurcation lesion in a patient. In this embodiment, the inflation lumen 34 inflates inflatable portions 28, 32 substantially simultaneously. In an alternative embodiment, branch balloon portions 27 and 30 have separate inflation lumens. In this alternative embodiment inflatable portions 28 and 32 can be inflated simultaneously or sequentially. When sequential inflation is desired, preferably, the first inflatable portion 28 is inflated first, followed by the inflation of the second portion 32.

First main guidewire lumen 22 extends through main vessel branch portion 18 and first inflatable portion 28. Although first guidewire lumen 22 extends through first inflatable portion 28 in the embodiment depicted in FIGS. 1-2, it is distinct from inflation lumen 34 and is not in fluid communication with the interior of balloon 26 as shown. Preferably, the first guidewire lumen 22 extends distally of first inflatable portion 28 and has an open distal end. Alternatively, guidewire lumen 22 can extend through branch portion 30.

In the embodiment depicted in FIGS. 1-2, an optional side sheath 20 is illustrated which does not include an inflatable balloon. Although in alternative embodiments side sheath 20 could include an inflatable portion, as described in further detail herein. Side sheath 20 is exterior to and distinct from inflation lumen 34 and thus is also not in fluid communication with the interior of balloon 26 as shown. As shown in the embodiment of FIGS. 1-2, side sheath 20 preferably extends distally of balloon 26, and may include a proximal open end 37 at any point along the length of the stent delivery system and a distal open end 39. Side sheath 20 can be of the type as described in U.S. Pat. No. 6,325,826 to Vardi, et al., for example, and in operation the side sheath 20 can extend through a branch access hole of the stent (see, e.g., FIG. 4).

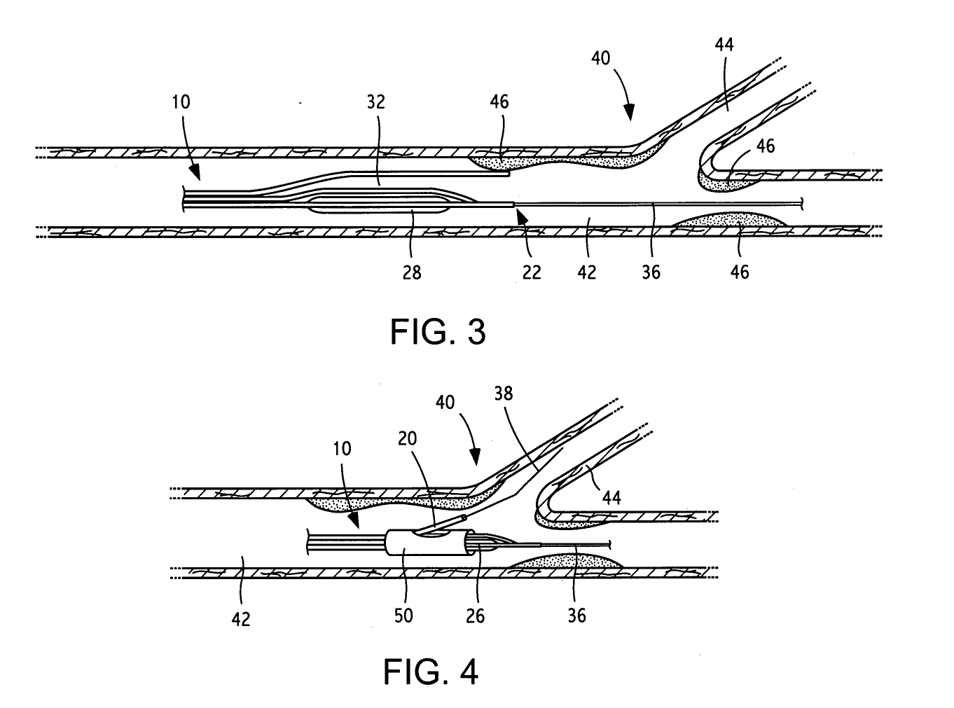

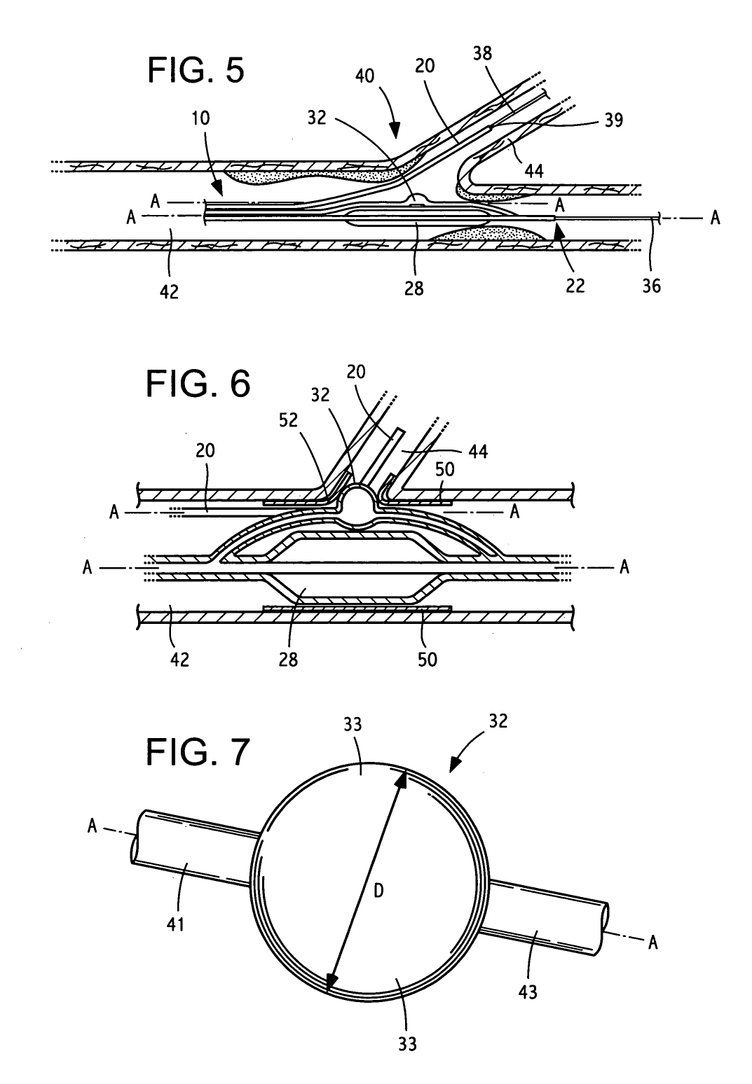

With reference to FIGS. 3-6, an exemplary manner of practicing the invention will now be discussed. Referring to FIGS. 3 and 5, the delivery system is shown in relation to an exemplary body lumen adjacent a blood vessel bifurcation 40 usually comprised of plaque and the delivery system 10 is shown without a stent mounted thereon (FIGS. 3 and 5). FIGS. 4 and 6 show the stent delivery system 10 with a stent 50 mounted thereon.

Bifurcation 40 includes a main vessel 42 and a branch vessel 44. Illustrative obstructions 46 located within bifurcation 40 may span or at least partially obstruct main vessel 42 and a proximal portion branch vessel 44. Generally, stent delivery system 10 may be threaded over a first main guidewire placed in the main vessel to guide the delivery system to the treatment site. More specifically, the proximal end of first guidewire 36 is threaded into the distal open end of the main guidewire lumen 22 and the delivery system is tracked to a position at or near bifurcation 40, as depicted in FIG. 3. Second guidewire 38 (FIG. 5) is then threaded into stent delivery system 10 from the proximal end of the delivery system. More specifically, second guidewire 38 is threaded into the open proximal end 37 of side sheath 20, and may extend therefrom through the open distal end 39 of side sheath 20, as depicted in FIG. 5. Alternatively, second guidewire 38 can be resting dormant on the inside of the side sheath, and when the system is proximal the bifurcation 40, it can be advanced out of side sheath 20 into side branch vessel 44. The systems in accordance with the principles of the invention may be used in over-the-wire or rapid exchange systems, which may include rapid exchange on either or both of the side sheath or main catheter. Rapid exchange is described in one exemplary embodiment in US2003/0181923 to Vardi et al., published Sep. 25, 2003, the entire contents of which are incorporated herein by reference.

In one embodiment, the stent delivery system 10 is positioned near bifurcation 40, and with the distal end 16 (FIG. 1) positioned near side branch vessel 44 (FIGS. 3-6), second guidewire 38 is advanced into side branch vessel 44 from side sheath 20. Then, the first and second inflatable portions of balloon 26 are positioned adjacent the opening of side branch vessel 44 such that auxiliary inflatable side portion 32 of bifurcated balloon 26 is aligned with side branch vessel. In one exemplary embodiment, alignment may be achieved using markers, as described in U.S. Pat. No. 6,692,483 to Vardi, et al., the entire contents of which is incorporated herein by reference. Second guidewire 38 remains in side branch sheath 20, and the distal end 16 of system 10 remains in main vessel 42. First guidewire 36 remains within first guidewire lumen 22, and may be further advanced and positioned in main branch vessel 42.

Once the system is properly positioned, pressurized fluid is supplied to first and second inflatable portions 28 and 32, respectively, of balloon 26 to dilate the body lumen and expand a stent mounted thereon (FIG. 6). Preferably, the inflatable portion 28 expands the main body of the stent and inflatable portion 32 expands the side (opening) and expandable branch structure of the stent, as discussed in more detail with reference to FIG. 6. After inflatable portions 28 and 32 have been inflated as described above, balloon 26 is deflated by draining the inflation fluid via inflation lumen 34. This allows the inflatable portions 28 and 32 to collapse in preparation for withdrawal of the assembly from vessel 42.

Referring now to FIGS. 4 and 6, one preferred embodiment is shown with stent delivery system 10 and an exemplary stent 50 mounted on the exterior of distal end 16 of the stent delivery system. Stent 50 includes an extendible branch portion 52 configured to extend into a branch vessel as will be discussed in further detail herein. The second inflatable portion 32 may be configured and positioned to deploy the outwardly expanding stent elements or branch portion 52 and may be positioned adjacent to the branch portion 52, or into a side branch access opening in the stent. As illustrated in FIG. 4, the second inflatable portion is preferably located radially within the outer periphery of the stent 50 prior to inflation. As shown in FIG. 6, when first and second inflatable portions 28 and 32 are expanded, they simultaneously or sequentially, depending upon the configuration of the inflation lumen, cause the stent 50 to expand in the main vessel 42 and the branch portion 52 of stent 50 to be pushed or extended into the branch vessel 44. Upon inflation of the balloon 26, the second inflatable portion 32 expands and extends the branch portion 52 toward the branch vessel to open and support the entrance or ostium of the side branch artery. This would occur simultaneously when the balloons share a common inflation lumen but could be sequentially inflated if separate inflation lumens are used. Although a bifurcated balloon is depicted, as shown, more than two inflatable portions or more than two balloons may be utilized with the present invention.

As illustrated, for example, in FIGS. 5 and 6, the first and second branch portions 27 and 30 have a longitudinal axis A. The longitudinal axies are substantially parallel with each other. The term “substantially parallel” is intended to encompass deviations from a purely parallel relationship which may be caused by flexure of the branch portions 27 and 30, or other components, experienced during insertion, travel, and deployment within a body lumen.

Various alternative balloon configurations will now be described which are designed to facilitate expansion of a branch structure portion of a stent.

FIG. 7 is an enlarged perspective view of the second balloon or auxiliary inflatable portion 32 of bifurcated balloon 26, which is referred to in the previous embodiments depicted in FIGS. 1-6. According to this embodiment, the central portion 33 of the auxiliary inflatable side portion 32 extends in a generally equidistant manner from the longitudinal axis A, and at an angle of up to about 90° relative to longitudinal axis A, but other angles are contemplated. As illustrated in FIG. 7, the auxiliary inflatable side portion 32 can have a generically spherical central portion 33 which is connected to a proximal shaft 41, as well a distal shaft 43. The components of the auxiliary inflatable side portion 32 may be sized appropriately, as will be readily apparent to those skilled in the art. The central spherical portion 33 can be provided with a suitable inflated diameter D. The diameter D can vary according to various factors known to those skilled in the art. According to a non-limiting, exemplary embodiment, the diameter D can be on the order of a few millimeters. For example, the diameter D is on the order of about 1.5-6.0 mm and, preferably, on the order of about 3.34-3.36 mm.

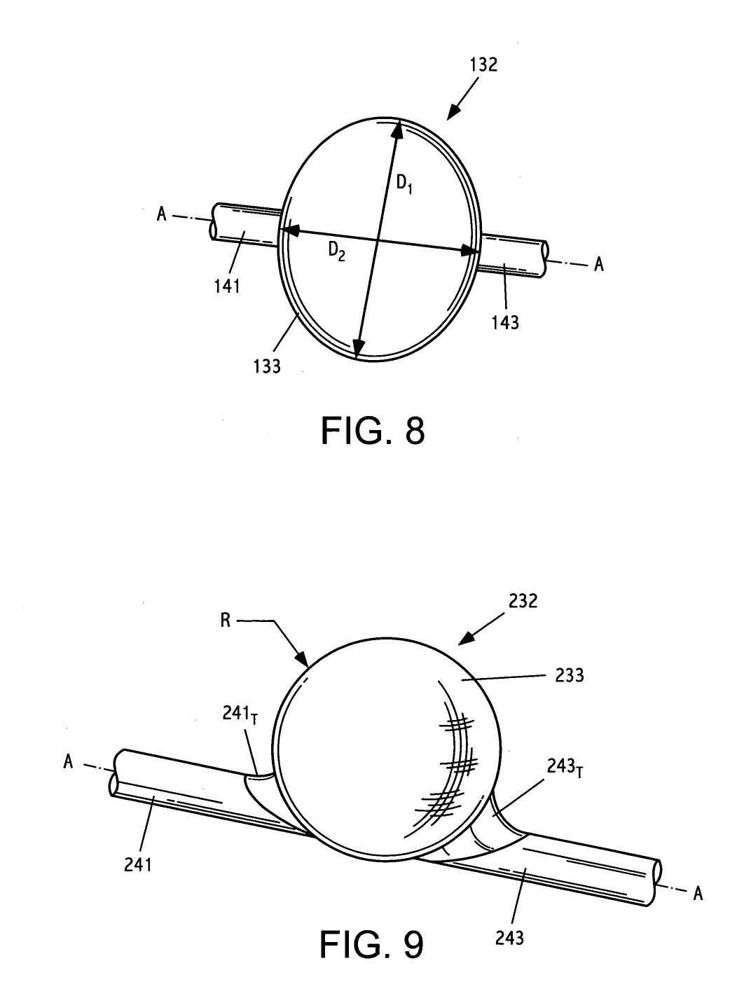

FIG. 8 illustrates an alternative second balloon or auxiliary inflatable side portion construction 132. According to this embodiment, the central portion 133 of the auxiliary inflatable side portion 132 extends in a generally equidistant manner from the longitudinal axis A, and at an angle of up to about 90° relative to longitudinal axis A, but other angles are contemplated. As illustrated in FIG. 8, the balloon 132 comprises a generally elliptical central portion 133, as well as a proximal shaft portion 141, and distal shaft 143 connected thereto. As with the previous embodiment, the various components of the balloon 132 may be sized as appropriate within appropriate dimensional ranges, as determined by those skilled in the art. The elliptical central section 133 of the balloon 132 is provided with major and minor diameters, D₁ and D₂, respectively, as illustrated in FIG. 7. According to non-limiting exemplary embodiments, the elliptical central section may be shaped such that the ratio D₂/D₁ is on the order of about 0.8. According to further exemplary non-limiting embodiments, the major diameter D₁ is preferably on the order of about 3.65-3.85 mm and can range from 1.5-6 mm, while the minor diameter D₂ is smaller than D₁ and is preferably on the order of about 2.9-3.1 mm.

FIG. 9 illustrates yet a further embodiment of a second balloon or auxiliary inflatable side portion 232 of bifurcated balloon 26 constructed according to the principles of the present invention. According to this embodiment, the central portion 232 is offset relative to the longitudinal axis A and preferably extends toward and/or into the branch vessel 44. The central portion 232 may extend at an angle of up to about 90° relative to longitudinal axis A, but other angles are contemplated. As illustrated in FIG. 9, the auxiliary inflatable side portion 232 of balloon 26 comprises an offset central bulbous or generally spherical portion 233, with a proximal shaft portion 241 and distal shaft portion 243 connected thereto via a proximal transition section 241_{T} and distal transition 243_{T}, respectively. As with the previous embodiments, the various components of the auxiliary inflatable side portion 232 of balloon 26 can be sized as appropriate, and as readily determined by those skilled in the art. According to exemplary, non-limiting embodiments, the auxiliary inflatable side portion 232 of balloon 26 can be configured such that the central offset portion 233 is provided with a radius of curvature R which is on the order of about 0.50-3.0 mm.

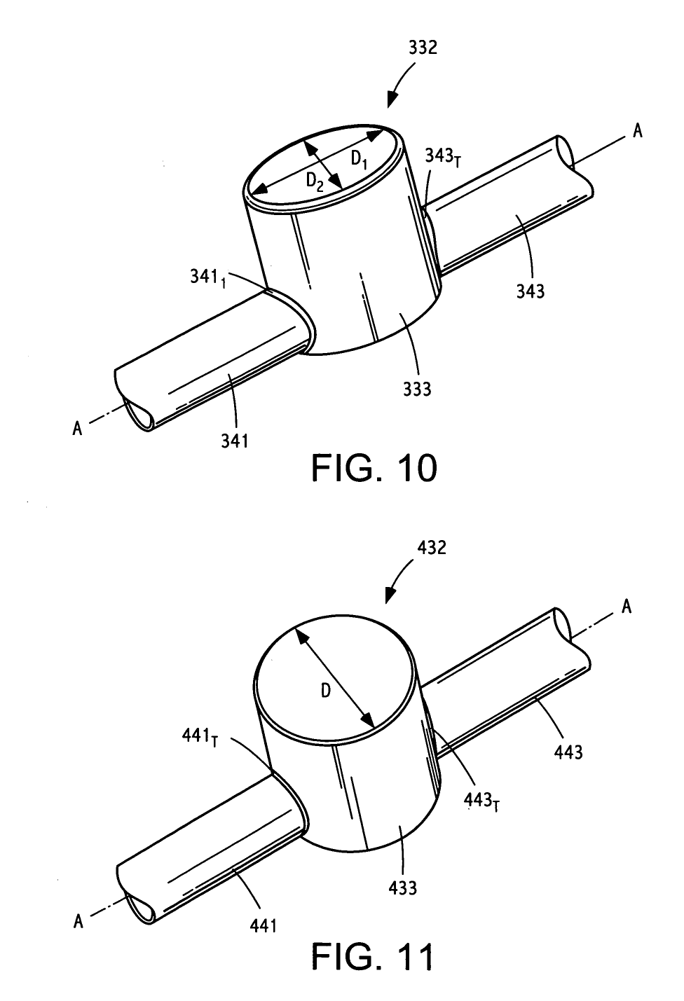

FIG. 10 illustrates yet another alternative embodiment for a second balloon or auxiliary inflatable side portion 332 of bifurcated balloon member 26. According to this embodiment, the central portion 332 is offset relative to the longitudinal axis A and preferably extends toward and/or into the branch vessel 44 (not shown). The central portion 332 may extend at an angle of up to about 90° relative to longitudinal axis A, but other angles are contemplated. As shown in FIG. 10, the auxiliary inflatable side portion 332 is configured such that it comprises a generally offset elliptical and cylindrical central section 333, with proximal shaft portions 341 and distal shaft portions 343 connected thereto via proximal transition section 341_{T} and distal transition portion 343_{T}, respectively. The offset central section 333 is preferably configured such that it comprised a first diameter D₁ and second diameter D₂ wherein D₁ and D₂ have different values (D1≠D2). The dimensions of the various constituent components of the auxiliary inflatable side portion 332 can be determined by those skilled in the art. According to exemplary non-limiting embodiments, the auxiliary inflatable side portion 332 can be configured such that it is provided with first and second diameters such that the ratio D₂/D₁ is on the order of about 0.25-4.0 mm. According to further, non-limiting examples, the auxiliary inflatable side portion 332 can be configured such that it is provided with a first diameter D₁ which has dimensions on the order of about 1.5-6.0 mm and, preferably about 2.7-2.9 mm, and a second diameter D₂ which has dimensions on the order of about 1.5-6.0 mm, and preferably about 2.1-2.3 mm.

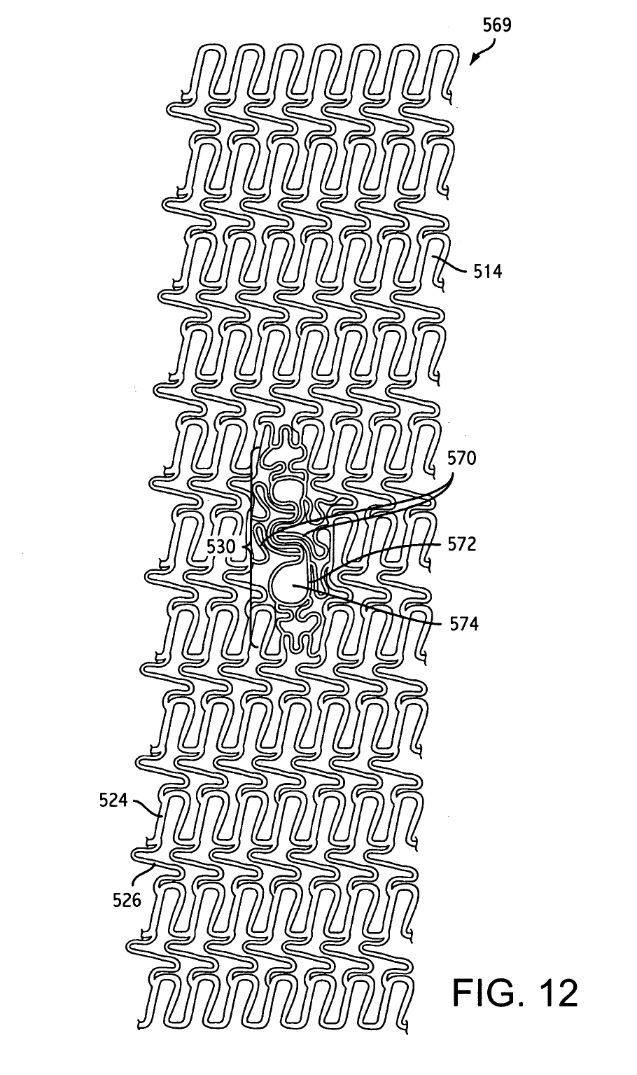

FIG. 11 illustrates yet another alternative embodiment of a second balloon or auxiliary inflatable side portion 432 of bifurcated balloon 26. According to this embodiment, the central portion 432 is offset relative to the longitudinal axis A and preferably extends toward and/or into the branch vessel 44 (not shown). The central portion 432 may extend at an angle of up to about 90° relative to longitudinal axis A, but other angles are contemplated. The auxiliary inflatable side portion 432 is configured such that it comprises an offset generally cylindrical central section 433 having a proximal shaft portion 441 and a distal shaft portion 443 connected thereto via proximal transition shaft portion 441_{T} and distal transition portion 443_{T}, respectively. The various constituent components of the balloon 432 can be configured with relative dimensions which can be ascertained by those skilled in the art. According to exemplary, non-limiting examples, the balloon 432 can be configured such that it is provided with an offset generally cylindrical central section 433 having a diameter D which is on the order of about 1.5-6.0 mm.

Various alternative stent constructions will now be described by reference to FIGS. 12-21.

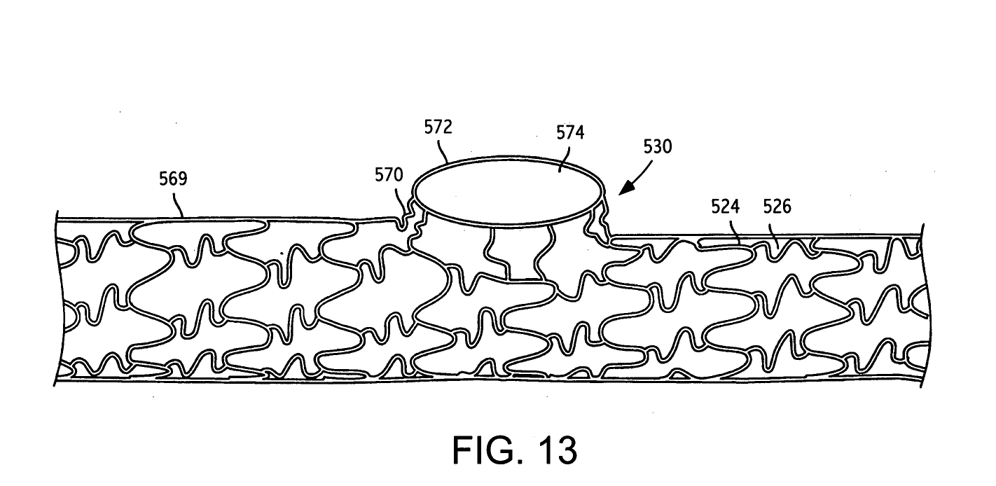

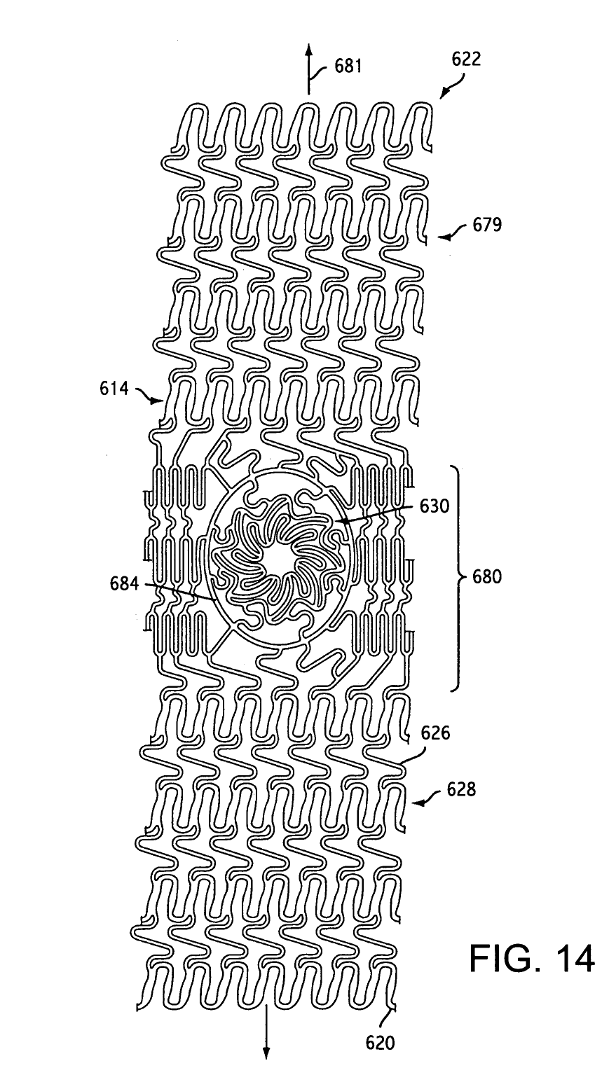

Referring now to FIGS. 12 and 13, an alternate embodiment of stent 569 is shown and includes an alternate branch portion 530. In this particular embodiment, branch portion 530 comprises support struts 570 and an expandable ring 572. Branch portion 530 defines at least one side opening 574. In one embodiment, the dimensions of the cell defining side opening 574 are such that the side opening 574 (prior to expansion of the stent) is larger than other openings in stent body 514. The presence of side opening 574 is generally configured to accommodate a side sheath therethrough and allow a physician to access a branch vessel during or after a procedure. In a particular embodiment, as shown in FIG. 12, side opening 574 is surrounded by expandable ring 572 of continuous material. In alternative embodiments, expandable ring 572 comprises unattached portions, or one portion that only partially covers side opening 574. A series of support struts 570 connect expandable ring 572 with struts 524 and connectors 526. Support struts 570 preferably comprise patterns in a folded or wrap-around configuration that at least partially straighten out during expansion, allowing expandable ring 572 to protrude into the branch vessel.

In this embodiment, when stent 569 is expanded, as shown in FIG. 13, branch portion 530 is extended into the branch vessel, causing expandable ring 574 to at least partially cover the inner surface of the branch vessel. Thus, in a preferred embodiment, the stent coverage in a portion the branch vessel includes the full circumference of the inner branch vessel wall. In alternative embodiments, partial coverage or several sections of coverage are present.

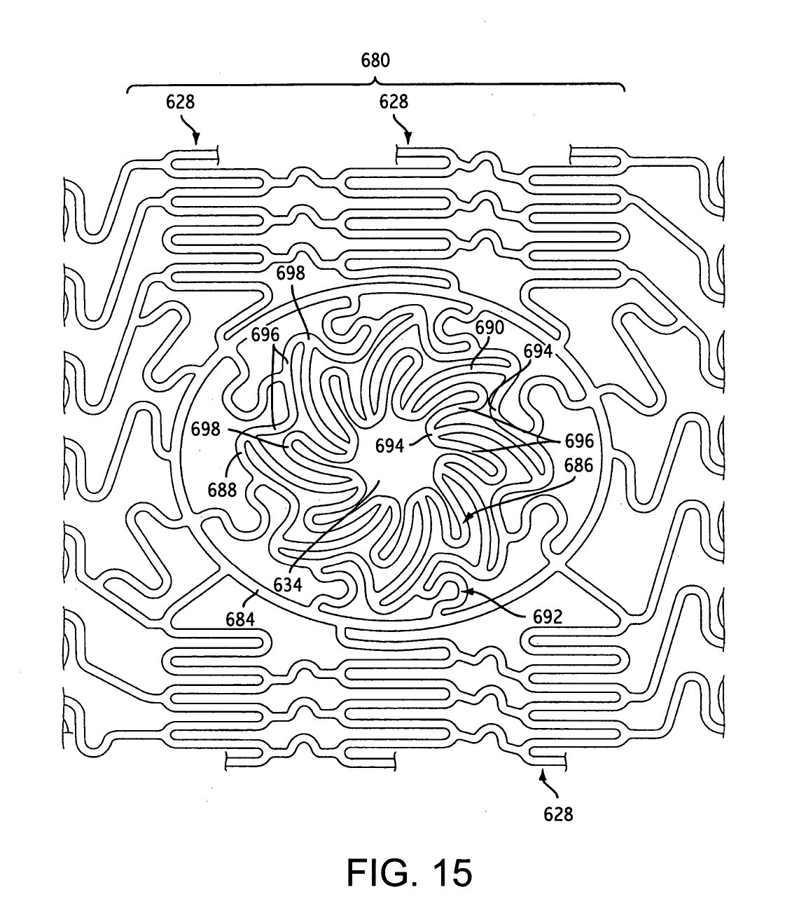

Referring to FIGS. 14-16, another embodiment of a stent 679 is shown having a main stent body 614 and another embodiment of a branch portion 630. FIGS. 14 and 15 show stent 679 in the unexpanded condition where branch portion 630 has not been deployed. FIG. 28 shows the stent 679 in the expanded configuration where the branch portion 630 has been expanded. As shown, main stent body 614 includes a main stent pattern having generally repeatable ring 628 and connector 626 pattern. Branch portion 630 and the surrounding midsection 680 interrupt the repeatable ring 628 and connector 626 pattern of stent 679. In this embodiment, branch portion 630 is configured to be both radially expandable and longitudinally extendable into the branch vessel and relative to its longitudinal axis 681 so that, in a preferred embodiment, the branch portion 630 contacts the entire periphery or circumference of the inner wall of the branch vessel in the expanded configuration. In this regard, branch portion 630 preferably provides 360° coverage of the wall of the branch vessel. That is, branch portion 630 can be extended outward with respect to longitudinal axis 681 of stent 679, and can also be expanded radially about axis 683 so as to contact the vessel (thereby allowing it to be adjustable with respect to vessel size).

Referring to FIG. 15, an enlarged view of section 680 of stent 679 is shown. In a preferred embodiment, a structural support member 684 may be provided as a transition between the main stent body 614 and branch portion 630. In one aspect of a preferred embodiment, structural support member 684 may be elliptical to accommodate branch vessels extending at an angle to the main vessel. In alternate embodiment, other shapes of support members 684 can be used to accommodate the vasculature. The structural support member 684 may include a continuous ring. In this embodiment, structural support member 684 is a full, non-expandable ring and it does not expand radially beyond a particular circumference.

As shown in FIGS. 14 and 15, two concentric rings, inner ring 686 and outer ring 688, are positioned within structural support member 684 and surround a generally circular branch opening 634 to provide access to the side branch vessel when stent 679 is in the unexpanded condition. Rings 686 and 688 are interconnected by a plurality of inner connectors 690. Outer ring 688 is connected to structural support member 684 by a plurality of outer connectors 692. Rings 686 and 688 are generally curvilinear members. For example, rings 686, 688 can be defined by undulation petals, prongs, or peaks 694. In a preferred embodiment, each ring 686, 688 have the same number of undulation peaks 694, but the inner ring may be more closely or tightly arranged, as shown. In another preferred embodiment, each ring 686, 688 has eight pedals or undulation peaks 694, although in alternate embodiments each ring can have any number of undulation peaks, and the number of peaks need not be equal for each ring. The undulation peaks 694 generally include a pair of strut portions 696 interconnected by curved portions 698, and the strut portions themselves are connected to adjacent strut portions by another curved portion. In a preferred embodiment, eight outer connectors 692 extend between structural support member 684 and outer ring 688, and each outer connector 692 is attached at one end to approximately the middle of a strut portion 696 of outer ring 688 and the structural support member 684 at the other end. As shown, outer connectors 692 may also have an undulated shape, although in alternate embodiments outer connectors 692 may have differing shapes. In another aspect of the preferred embodiment, outer connectors 692 may be evenly or symmetrically spaced about the structural support member 684. The inner ring 686 is attached to the outer ring 688 by a plurality of inner connectors 690 and, in a preferred embodiment; eight inner connectors 690 connect the rings. Inner connectors 690 extend from curved portion 698 of outer ring 688 to curved portion of inner ring 686. As shown in FIG. 15, in a preferred embodiment, inner connectors 690 have simple curved shape. Other qualities, configurations, sizes and arrangements of connectors, rings and spacing can be used depending upon the desired results. Varying the connectors can provide for different amounts of flexibility and coverage. The type of configuration of rings and connectors shown addresses the need for radial and longitudinal expansion of branch portion 630, as well as branch vessel coverage. Other configurations and arrangements for the branch portion can be used in accordance with the invention.

Referring again to FIGS. 14 and 15, the stent pattern surrounding branch portion 630 may be modified with a different pattern to accommodate branch portion 630, as can all of the aforementioned embodiments. In particular, the rings 628 in the midsection 680 may be configured and dimensioned to be denser to provide sufficient coverage and flexibility to composite for the area occupied by branch portion 630.

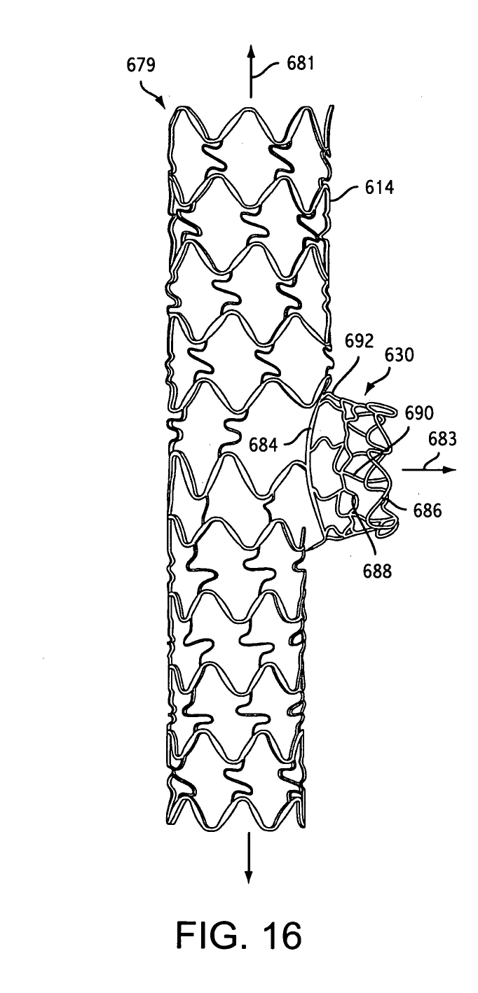

Referring now to FIG. 16, stent 679 is shown in the expanded configuration, with branch portion 630 deployed. Upon expansion of branch portion 630, the inner and outer rings 686, 688 shift about the longitudinal branch axis 683 and expand laterally away from the main stent body 614 and into the branch vessel to form a branch coverage portion. Upon expansion, the outer connectors 692 can move outwardly and the inner connectors 690 can straighten to a position substantially parallel to longitudinal branch axis 681. In a preferred embodiment, the expanded rings 686, 688 have substantially the same expanded diameter, although in alternate embodiments rings 686, 688 could also have different diameters to accommodate a tapered vessel, if, for example a tapered balloon is used. The branch portion 630 can be extended at different angles to the longitudinal axis 681 of the stent depending upon the geometry of the branch vessel being treated. In this embodiment, the branch portion 630 may preferably extend into the branch vessel about 1.5-3 mm.

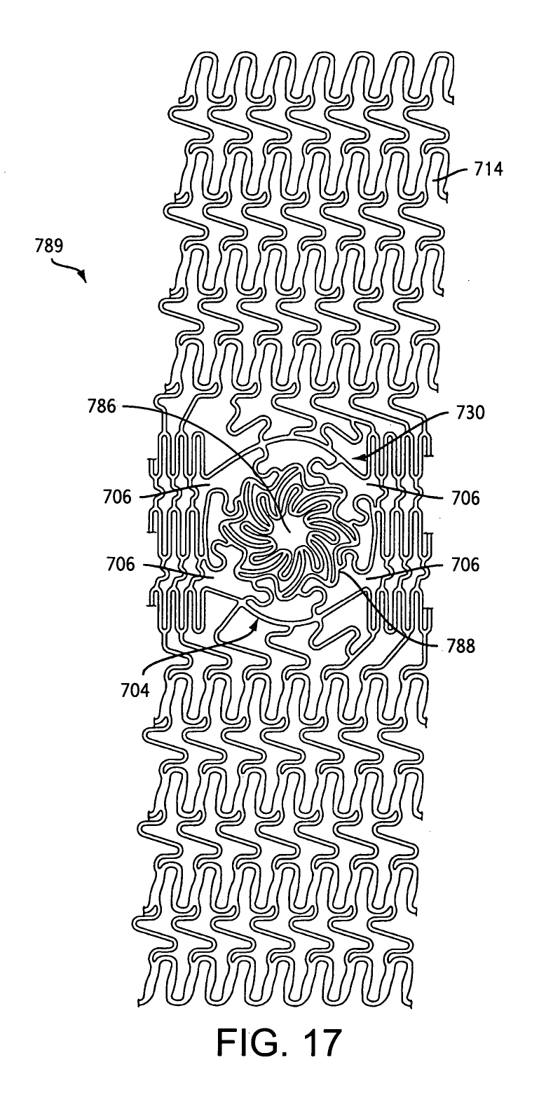

Referring now to FIG. 17, another embodiment in the form of a stent 789 is shown having a main stent body 714 and another embodiment of a branch portion 730. Stent 789 is substantially similar to stent 679, except 789 has a discontinuous support member 704 surrounding a two concentric ring 786, 788 structure. Support member 704 has a generally elliptical shape and includes a plurality of discontinuities 706 along the perimeter. The configuration of the discontinuous support member facilitates additional flexibility of the branch portion during expansion and generally provides for accommodating a greater range of branch vessel geometries. In one aspect of a preferred embodiment, structural support member 784 may be elliptical to accommodate branch vessels extending at an angle to the main vessel.

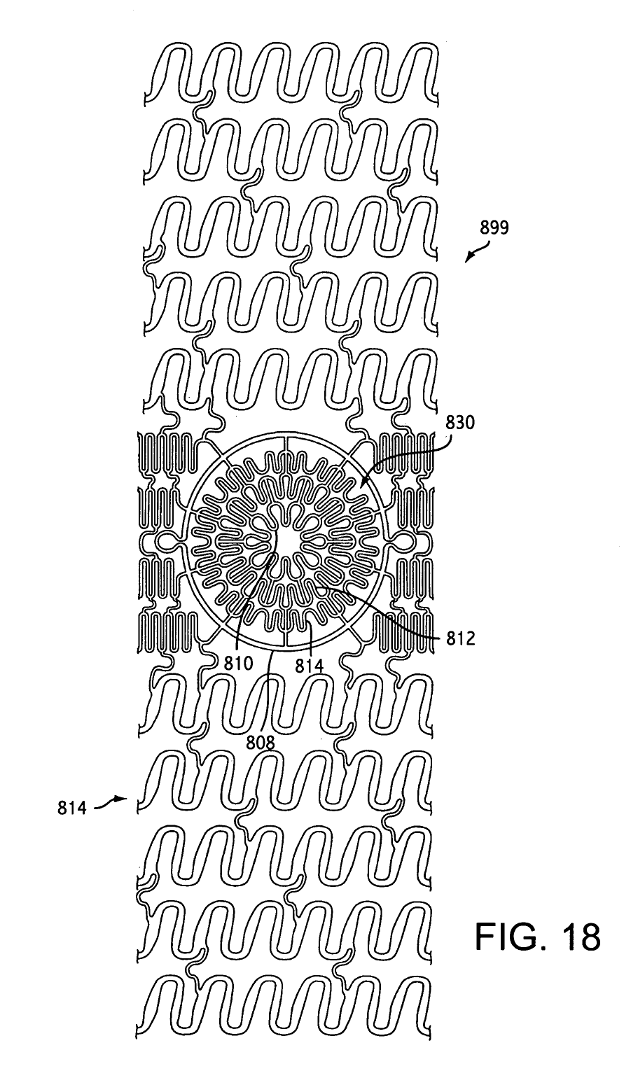

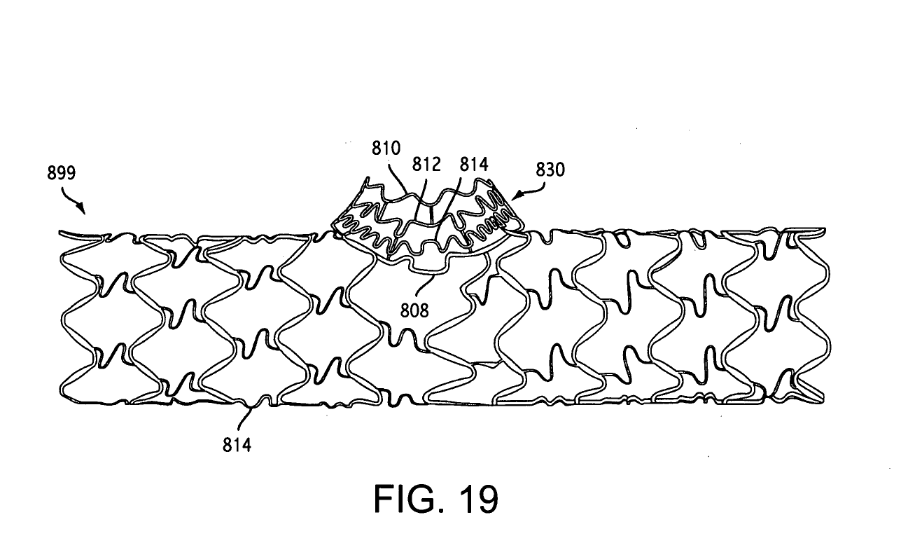

Referring to FIGS. 18 and 19, another embodiment of a stent 899 is shown in the unexpanded and expanded states, respectively. Stent 899 comprises a main stent body 814 and another embodiment of a branch portion 830. Stent 899 is substantially similar to stent 879, except stent 899 has a branch portion 830 including a support member 808 surrounding three concentric rings 810, 812, 814 instead if two. As can be seen in FIG. 19, when stent 899 is expanded the three concentric ring structure of this embodiment facilitates additional branch wall support because a generally more dense pattern is created in branch portion 830 with the addition of another concentric ring.

In all of the above embodiments, the branch portion protrudes into the branch vessel when the stent is fully expanded. The branch portion upon expansion can extend into the branch vessel in different lengths depending upon the application. The amount of extension may vary in a range between about 0.1-10.0 mm. In one preferred embodiment, the length of extension is 1-3 mm. In another preferred embodiment, the length of extension is approximately 2 mm. In alternative embodiments, the amount of extension into the branch vessel may be variable for different circumferential segments of the branch portion. As shown in each of the embodiments, the branch portion is approximately 2.5 mm in width and about 2.5-3.0 mm in length. However, the branch portion can be dimensioned to accommodate varying size branch vessels. The branch portion can be formed of any tubular shape to accommodate the branch vessel, including, oval or circular, for example.

In all of the above embodiments, it should be understood that it is within the scope of the present invention to provide the stent with a configuration such that the proximal end of the stent is expandable to a greater or lesser degree than the distal end of the stent. For example, the stent, when expanded, may be constructed such that its outer diameter at the proximal end thereof is greater than the outer diameter at the distal end of the stent.

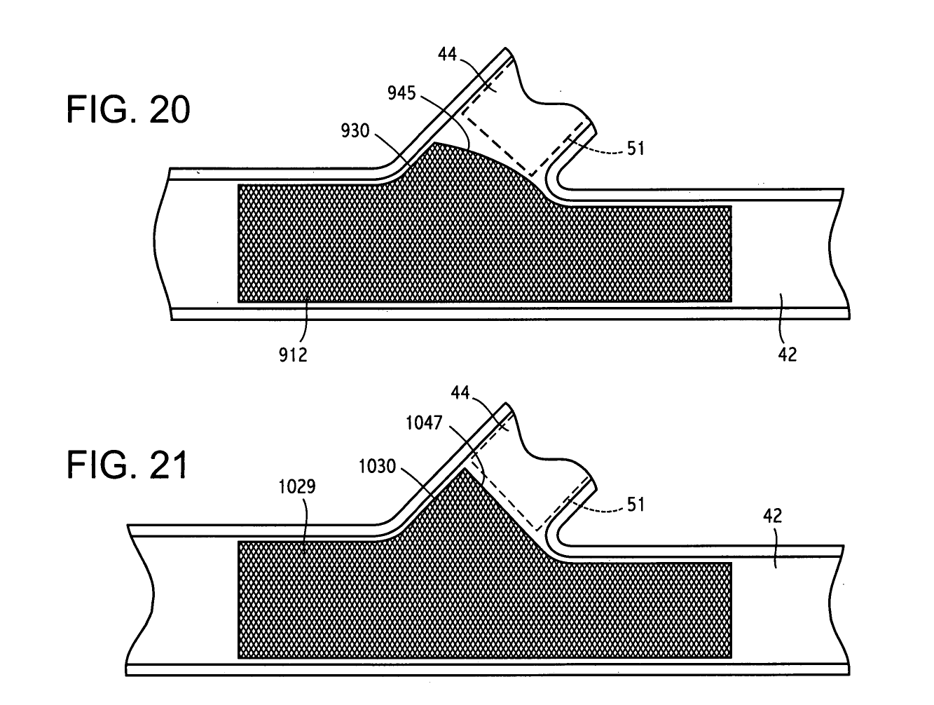

Referring to FIGS. 20 and 21, schematic views are shown of stents 912, 1029 in the expanded state as implemented at a blood vessel bifurcation. As shown in FIG. 20, stent 912 has a generally curved or radial profile along the distal perimeter 945 of branch portion 930 as it extends into branch vessel 44. The generally curved or radial profile is due to the particular geometry of branch portion 930 of stent 912. Referring to FIG. 21, stent 1029 has a generally tapered, straight or linear profile along the distal perimeter 1047 of the branch portion 1030 of the stent as it extends into branch vessel 44. The generally straight or linear profile in FIG. 21 is a result of the particular geometry of branch portion 1030 of stent 1029. In a preferred embodiment, the linear profile is at a right angle with respect to the axis of branch vessel 44. In alternative embodiments, however, the linear profile may be at any angle with respect to the axis of branch vessel 44. One advantageous feature of the linear profile along the distal perimeter of branch portion 1030 shown in FIG. 21 is that if a second stent 51 were to be used in branch vessel 44, the linear profile facilitates better alignment with the second stent and permits coverage of a larger surface area of the branch vessel wall. For example, if a second stent 51 were to be used in combination with stent 912 of FIG. 20, gaps may exist between the two stents at the interface between the radial distal perimeter 945 and an abutting straight or linear edge of a second stent, whereas a close abutting interface may be achieved with stent 1029 of FIG. 21.

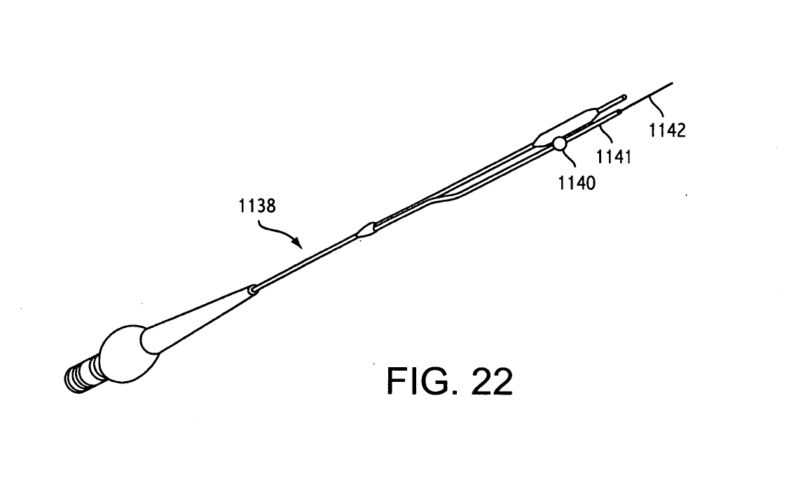

The balloon delivery systems and deployment methods of the previously described embodiments may he used with any of the aforementioned stent configurations. According to a further embodiment, the balloon configured to extend or expand the branch portion of the stent is located on the side sheath of the delivery system, such as the system 1138 depicted in FIG. 22. In this case, the system is a two-balloon system. As illustrated in FIG. 22, the second balloon is located such that the side sheath 1141 extends distally beyond the second balloon 1140. The second balloon 1140 can be positioned within a stent in a manner similar to that previously described herein and is preferably located radially within the stent prior to inflation. The side sheath 1141 may have an inflation lumen and a lumen for receiving a guidewire 1142 for locating the branch vessel 44. The second balloon 1140 may have a lumen for receiving a guidewire for locating the branch vessel. The second balloon may be located at any position along the length of the main balloon. For example, it can be located between proximal and distal ends of the stent, more particularly it can be located on the middle ⅓ of the stent. When employed on the side sheath, the second or auxiliary balloon 1140 can have the same shape or geometry as any of the previously described embodiments contained herein, such as those depicted in connection with FIGS. 6-11. In this regard, the proximal and distal shaft portions 41, 43, 141, 143, 241, 243, 341, 343, 441 and 443 of the balloon constructions illustrated in FIGS. 7-11 can be shaft portions of the side sheath 1141. Moreover, any of the previously described stent configurations may also be used in combination with the system 1138.

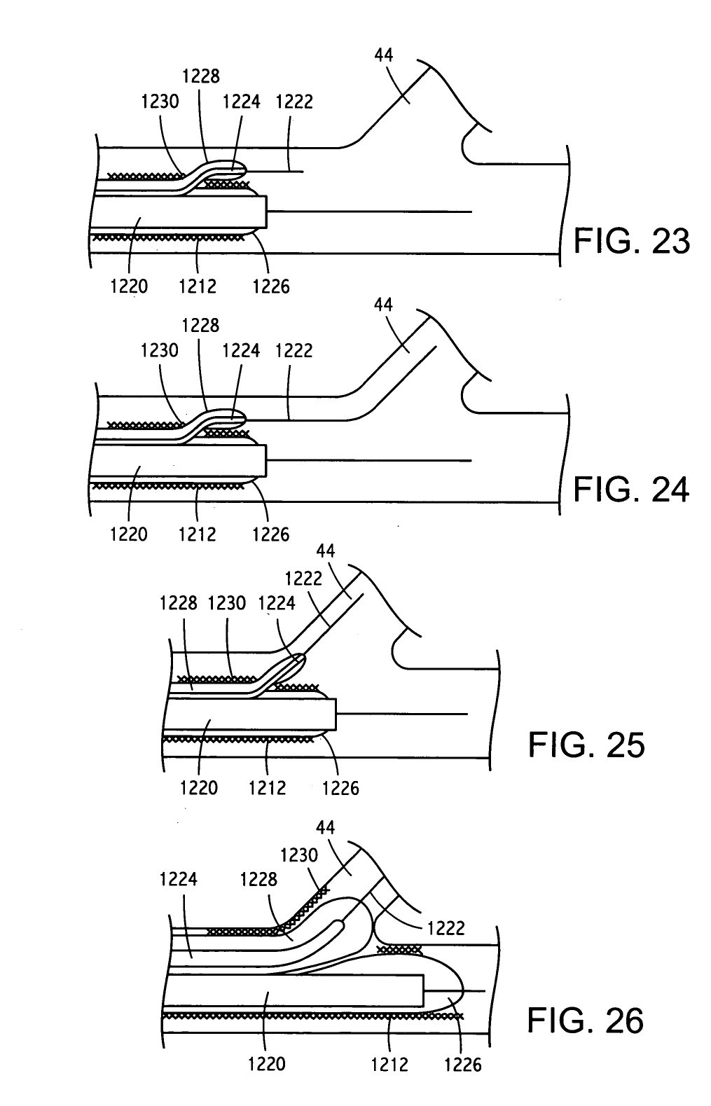

Referring now to FIGS. 23-26, illustrations of the steps of one alternative example of a method for employing a stent according to the invention are shown. By way of example, the method is depicted utilizing stent 1212. Methods for positioning such a catheter system within a vessel and positioning such a system at or near a bifurcation are described more fully in co-pending U.S. patent application Ser. No. 10/320,719 filed on Dec. 17, 2002, which is incorporated herein by reference in its entirety. As shown in FIG. 23, a catheter system 1220 is positioned proximal to a bifurcation, using any known method. A branch guidewire 1222 is then advanced through an opening in the stent and into the branch vessel 44, as shown in FIG. 24. In a preferred embodiment, the opening may be a designated side branch opening, such as an opening formed by the absence of some connectors, as described above. Branch portion 1230 is adjacent the opening. As shown in FIG. 25, if the side sheath 1224 is attached to the main catheter 1220, the main catheter 1220 is advanced along with the side sheath 1224. Alternatively, if the side sheath 1224 is separate from to the main catheter 120, the second catheter or side sheath 1224 is then advanced independently through the opening in the stent and into the branch vessel. Branch portion 1230 is positioned over a portion of the lumen of the branch vessel 44 as the side sheath 1224 is inserted into branch vessel 44. Referring to FIG. 26, a first balloon 1226 located on main catheter 1220 is then expanded, causing expansion of the stent body, and a second balloon 1228 located on the side sheath 1224 is also expanded, causing branch portion 1230 to be pushed outward with respect to the stent body, thus providing stent coverage of at least a portion of the branch vessel 44. The balloons are then deflated and the catheter system and guidewires are then removed.

One particular application for the use of a stent with a branch portion 30 such as the one described above is for localizing drug delivery. As discussed herein, restenosis, including in-stent restenosis, is a common problem associated with medical procedures involving the vasculature. Pharmaceutical agents have been found to be helpful in treating and/ or preventing restenosis, and these are best delivered locally to the site of potential or actual restenosis, rather than systemically.

While the invention has been described in conjunction with specific embodiments and examples thereof, it is evident that many alternatives, modifications and variations will be apparent to those skilled in the art upon reading the present disclosure. Accordingly, it is intended to embrace all such alternatives, modifications and variations that fall within the spirit and broad scope of the appended claims. Furthermore, features of each embodiment can be used in whole or in part in other embodiments.

1. A system for treatment of a bifurcated body lumen, the bifurcated body lumen comprising a main vessel and a branch vessel, the system comprising:
a catheter comprising a main catheter shaft and a first balloon associated with the main catheter shaft;
a side sheath and a second balloon associated with the side sheath; and
a stent comprising a generally cylindrical body having a proximal end and a distal end, a branch portion, and a branch access opening;
wherein the stent is positioned relative to the side sheath such that the first balloon is adapted to expand the main body portion of the stent, and the second balloon is adapted to extend the branch portion toward the branch vessel, and the second balloon is longitudinally located between the proximal end and the distal end of the stent; and
wherein at least a portion of the side sheath extends through the branch access opening.

2. The system of claim 1, wherein the main catheter shaft and the side sheath comprise a proximal end and a distal end, wherein the main catheter shaft and the side sheath are connected at the proximal end, and are separate at the distal end.

3. The system of claim 1, wherein the main catheter shaft and the side sheath are separate members.

4. The system of claim 1, wherein the side sheath extends distally beyond the second balloon.

5. The system of claim 1, further comprising a first inflation lumen associated with the first balloon and a second inflation lumen associated with the second balloon.

6. The system of claim 5, wherein the first and second inflation lumens are not in fluid communication with each other.

7. The system of claim 1, wherein the main catheter shaft comprises a guidewire lumen for passage of a guidewire to locate the catheter within the main vessel.

8. The system of claim 7, wherein the side sheath comprises a guidewire lumen for passage of a guidewire to locate the side sheath within the branch vessel.

9. The system of claim 1, wherein the inflatable portion of the second branch portion is generally spherical.

10. The system of claim 1, wherein the inflatable portion of the second branch portion is generally elliptical and comprises a major and minor axis

11. The system of claim 1, wherein the inflatable portion of the second branch portion is generally in the form of an offset bulbous shape.

12. The system of claim 1, wherein the inflatable portion of the second branch portion is generally in the form of an offset elliptical cylinder.

13. The system of claim 1, wherein the inflatable portion of the second branch portion is generally in the form of an offset cylinder.

14. The system of claim 1, the stent further comprising a branch access opening, and the branch portion comprises an outwardly expandable portion disposed around any portion of the branch access opening, wherein expanding the second balloon deploys the outwardly expandable portion of the stent toward the branch vessel.

15. The system of claim 1, wherein the generally cylindrical body of the stent comprises a geometrical configuration defining a first pattern comprising a pattern of struts and connectors, and the branch portion comprises a geometrical configuration defining a second pattern.

16. The system of claim 15, wherein the second pattern comprises a pattern of struts and connectors, and comprises a portion having at least one missing connector in the pattern.

17. The system of claim 16, wherein the portion has a plurality of missing connectors.

18. The system of claim 15, wherein the second pattern comprises a pattern of struts and connectors, and wherein the struts of the second pattern are more densely packed than the struts in the first pattern.

19. The system of claim 15, wherein the struts in the first pattern have a first length, and the struts in the second pattern have a second length, and wherein the first length is different than the second length.

20. The system of claim 15, wherein the struts in the first pattern have a first density, and the struts in the second pattern have a second density, and wherein the first density is different than the second density.

21. The system of claim 1, wherein the second balloon is longitudinally located in the middle one-third of the stent.

22. The system of claim 1, wherein the generally cylindrical body of the stent defines an outer perimeter, wherein the second balloon is located radially inward of the outer perimeter when the second balloon is not inflated.

23. The system of claim 1, wherein the proximal end of the stent is constructed such that it is expandable to a greater outer diameter than the distal end of the stent.

24. A system for treatment of a bifurcated body lumen, the bifurcated body lumen comprising a main vessel and a branch vessel, the system comprising:
a catheter comprising a main catheter shaft and a first balloon associated with the main catheter shaft;
a side sheath and a second balloon associated with the side sheath; and
a stent comprising a generally cylindrical body defining an outer perimeter having a proximal end and a distal end and a branch portion;
wherein the stent is positioned relative to the side sheath such that the first balloon is adapted to expand the main body portion of the stent, and the second balloon is adapted to extend the branch portion toward the branch vessel, and wherein the second balloon is located radially inward of the outer perimeter when the second balloon is not inflated.

25. The system of claim 24, wherein the branch portion of the stent comprises a branch access opening.

26. The system of claim 25, wherein at least a portion of the side sheath extends through the branch access opening.

27. The system of claim 24, wherein the first balloon and the second balloon are located between the proximal end and the distal end of the stent.

28. The system of claim 24, wherein the second balloon is longitudinally located in the middle one-third of the stent.

29. The system of claim 24, wherein the proximal end of the stent is constructed such that it is expandable to a greater outer diameter than the distal end of the stent.

30. The system of claim 24, wherein the proximal end of the stent is constructed such that it is expandable to a greater outer diameter than the distal end of the stent.

31. A method for treating a bifurcated body lumen, the bifurcated body lumen comprising a main vessel and a branch vessel, the method comprising:
(i) advancing a catheter system through the main vessel, the catheter system comprising:
a main catheter shaft and a first balloon associated with the main catheter shaft;
a side sheath and a second balloon associated with the side sheath; and
a stent comprising a generally cylindrical body having a proximal end, a distal end, a branch portion, and a branch access opening;
wherein at least a portion of the side sheath extends through the branch access opening; and wherein the second balloon is longitudinally located between the proximal end and the distal end of the stent;
(ii) positioning the branch portion of the stent proximate to the branch vessel;
(iii) inflating the first balloon thereby causing expansion of the generally cylindrical body of the stent; and
(iv) inflating the second balloon thereby causing the branch portion of the stent to be pushed outward with respect to the generally cylindrical body of the stent.

32. The method of claim 31, wherein the main catheter shaft and the side sheath comprise a proximal end and a distal end, wherein the main catheter shaft and the side sheath are connected at the proximal end, and are separate at the distal end.

33. The method of claim 31, wherein the main catheter shaft and the side sheath are separate members.

34. The method of claim 31, wherein steps (iii) and (iv) are performed simultaneously.

35. The method of claim 32, wherein steps (iii) and (iv) are performed sequentially.

36. The method of claim 31, wherein the first balloon and the second balloon are located between the proximal end and the distal end of the stent.

37. The method of claim 32, wherein at least one of steps (i) and (ii) comprise advancing the catheter system over at least one guidewire.

38. The method of claim 31, further comprising advancing at least a portion of the side sheath into the branch vessel.

39. The method of claim 31, wherein the expansion of the second balloon in step (iv) causes the branch portion of the stent to cover at least a portion of the branch vessel.

40. The method of claim 31, further comprising:
(v) deflating the first and second balloons; and
(vi) removing all components of the catheter system from the main and branch vessels, except for the stent.

41. The method of claim 31, wherein the second balloon is longitudinally located in the middle one-third of the stent.

42. The method of claim 31, wherein the generally cylindrical body of the stent defines an outer perimeter, wherein the second balloon is located radially inward of the outer perimeter when the second balloon is not inflated.

43. The method of claim 31, wherein step (iii) comprises expanding the proximal end of the stent to a greater degree than the distal end of the stent.
